(19) United States
(12) Patent Application Publication
(10) Pub. No.: US 2025/0129006 A1
(43) Pub. Date: Apr. 24, 2025

(54) PRODUCTION OF BENZOIC ACID FROM A BIOMASS

(71) Applicant: SYENSQO SA, Brussels (BE)

(72) Inventors: Jianxia Zheng, Shanghai (CN); Stéphane Streiff, Lyon (FR); Sergio Mastroianni, Lyon (FR)

(73) Assignee: SYENSQO SA, Brussels (BE)

(21) Appl. No.: 18/681,459

(22) PCT Filed: Aug. 5, 2021
(86) PCT No.: PCT/CN2021/110781

§ 371 Date: Feb. 5, 2024

(51) Int. Cl. C07C 51/235; C07C 37/16; C07C 45/51; C12P 7/06

(52) U.S. Cl. CPC C07C 51/235; C07C 37/16; C07C 45/512; C12P 7/06; C07C 2523/644

(57) ABSTRACT

Process for the production of benzoic acid, said process comprising the steps of: (A) producing ethanol from a bio-mass, which is advantageously a carbohydrate-containing biomass, then (B) causing the ethanol produced at the step (A) to react with methanol, so as to produce benzyl alcohol and/or benzaldehyde, then (C) causing the benzyl alcohol and/or benzaldehyde produced at the step (B) to react with oxygen, so as to produce benzoic acid.

### TECHNICAL FIELD

The present invention relates to the production of benzoic acid from a biomass.

### BACKGROUND ART

Benzoic acid (C₆H₅COOH) is an aromatic chemical of high industrial interest. As such, it can be used as expectorant, as analgesic, as antiseptic or as antimicrobial agent to preserve foods, beverages, toothpastes, mouthwashes, dentifrices, cosmetics and pharmaceuticals. Even if benzoic acid is commonly used as antimicrobial agent, it is often substituted in this application by its salts which exhibit a much higher solubility in water, especially sodium benzoate. Benzoic acid and esters thereof, especially methyl benzoate, are also widely used for the production of benzoate plasticizers, especially diethylene and dipropylene glycol dibenzoates. Benzoic acid can also be used for the manufacture of other useful chemicals such as ε-caprolactam.

At the industrial scale, most of the benzoic acid is produced by the oxidation of petroleum-based toluene with molecular oxygen. Typically, petroleum-based toluene is oxidized with molecular oxygen in the liquid phase using a heavy metal catalyst such as a cobalt or manganese naphthenate. Processes based on the oxidation of toluene suffer from various technical drawbacks, including corrosion issues in the reactor, high energy consumption and the possible formation of many by-products such as carbon monoxide, carbon dioxide, formic acid, acetic acid, biphenyls, monomethyl biphenyls, biphenyl monocarboxylic acids and high-boiling tar like materials. Having said this, the paramount issue with these processes is undoubtedly intrinsic to the main starting reagent itself, viz. toluene, which originates from a petroleum feedstock.

To address this issue, several researchers, essentially academics, have explored various alternative routes for benzoic acid production from renewable resources, especially from biomass.

One possible option for the production of benzoic acid from biomass is the use of the lignin fraction of lignin-containing biomasses (such as wood), which are the most abundant renewable sources. Many scientific publications describe the decomposition of lignin model based compounds into benzoic acid with different degrees of success. Among others, it can be referred to: (i) Yang Y. et al., Chemical Communications, 2015, 51, 4028; (ii) Deng W. et al., Green Chemistry, 2015, 17, 5009; (iii) Kim S. et al., ChemSusChem, 2016, 9, 241; (iv) Yang Y. et al., Chemical Communications, 2017, 53, 8850; (v) Liu X. et al., Acta Chimica Sinica, 2017, 75, 783; and (vi) Li H. et al., ChemSusChem, 2020, 13, 4660. None of them addresses the real industrial challenge of converting of a true lignin-containing biomass, in its whole complexity, into benzoic acid, and their interest has remained purely academic so far.

Another possible option for the production of benzoic acid from biomass is the use of carbohydrate fraction of a carbohydrate-containing biomass such as sugarcane. With this regard, a few publications describe the use of a carbohydrate susceptible of being obtained from a biomass for producing either benzoic acid or benzoate: on the one hand, Noda S. et al., Microbial Cell Factories, 2012, 11, 49 describes the production of benzoic acid from glucose, cellobiose, starch or cellulose in the presence of Streptomyces maritimus; on the other hand, Otto M. et al., Biotechnol. J., 2020, 15, 2000211 describes the use of glucose or glycerol for producing benzoate in the presence of an engineered Pseudomonas taiwanensis. A few other publications describe the use of a compound susceptible of being directly or indirectly obtained from a carbohydrate for the manufacture of benzoic acid; so are (i) Mahmoud E. et al., ACS Catalysis, 2015, 5, 6946, wherein furan is reacted with methyl acrylate to form benzoic acid, and (ii) Pfennig T. et al., Green Chemistry, 2017, 19, 4879, wherein coumalic acid is reacted with ethylene to form benzoic acid. Again, none of these publications addresses the real industrial challenge of converting of a true carbohydrate-containing biomass, in its whole complexity, into benzoic acid, and their interest has remained purely academic so far.

There is a need for a process which is fully capable of producing benzoic acid from real biomass, in its whole complexity, with a reasonably high yield, and which has an excellent viability at industrial scale.

### SUMMARY OF THE INVENTION

This need is now met by the present invention.

The present invention concerns a process for the production of benzoic acid, said process comprising the steps of:
(A) producing ethanol from a biomass, then
(B) causing the ethanol produced at the step (A) to react with methanol, so as to produce benzyl alcohol and/or benzaldehyde, then
(C) causing the benzyl alcohol and/or benzaldehyde produced at the step (B) to react with oxygen, so as to produce benzoic acid.

These and other features, aspects and advantages of the present subject matter will be better understood with reference to the following description and appended claims.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively and any and all combinations of any or more of such steps or features.

### Definitions

For convenience, before further description of the present disclosure, certain terms employed in the specification, and examples are collected here. These definitions should be read in the light of the remainder of the disclosure and understood in the same way as a person of skill in the art understands them. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “and/or” includes the meanings “and” and “or”.

The terms “comprise”, “include”, “comprising” and “including” are used in the inclusive, open sense, meaning that additional elements may be included. Throughout this specification, unless the context requires otherwise, these terms and variations thereof, such as “comprises”, will be understood to imply the inclusion of a stated element or step or group of element or steps but not the exclusion of any other element or step or group of element or steps. The term “including” is used to mean “including but not limited to”. “Including” and “including but not limited to” are used interchangeably.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also all the individual numerical values or sub-ranges encompassed within that range as if each numerical value and sub-range is explicitly recited. For example, a temperature range of from 100° C. to 200° C. should be interpreted to include not only the explicitly recited range of 100° C. to 200° C., but also sub-ranges, such as 110° C. to 170° C., 120° C. to 160° C., and so forth, as well as individual amounts, including fractional amounts, within the specified ranges, such as 135° C. and 145.5° C.

In a first aspect, the present invention provides a process for the production of benzoic acid, said process comprising the steps of:
(A) producing ethanol from a biomass, then
(B) causing the ethanol produced at the step (A) to react with methanol, so as to produce benzyl alcohol and/or benzaldehyde, then
(C) causing the benzyl alcohol and/or benzaldehyde produced at the step (B) to react with oxygen, so as to produce benzoic acid.

According to the step (A), ethanol is produced from a biomass.

The biomass which is used in the process in accordance with the present invention is advantageously a carbohydrate-containing biomass. The carbohydrate content of the carbohydrate-containing biomass is usually of at least 2 wt %, very often at least 4 wt %, desirably at least 8 wt % and sometimes of at least 12 wt % or at least 16 wt %, based on the total weight of the carbohydrate-containing biomass. The carbohydrate-containing biomass can be natural or chemically modified. The carbohydrate contained in the carbohydrate-containing biomass can be (i) a sugar, in particular a monosaccharide (such as glucose, galactose, fructose or xylose), a disaccharide (such as sucrose, lactose or maltose) or a polyol (such as sorbitol or mannitol); (ii) an oligosaccharide such as a maltodextrin; (iii) a polysaccharide, in particular a starch (such as amylose or amylopectin) or a non-starch polysaccharide (such as glycogen, cellulose or hemicellulose); it is preferably a sugar, more preferably a monosaccharide (such as glucose, galactose, fructose or xylose). Thus, in a preferred embodiment, the biomass is a sugar-containing biomass, such as sugarcane, sugar beet, sweet sorghum and molasses; the sugar content of the sugar-containing biomass is usually of at least 2 wt %, very often at least 4 wt %, desirably at least 8 wt % and sometimes of at least 12 wt % or at least 16 wt %, based on the total weight of the sugar-containing biomass. An especially preferred biomass is a sugarcane. As herein used, the term “sugarcane” denotes both unmodified, natural sugarcane and energy canes, which are genetically modified sugarcanes to become more productive in the production of bioethanol, such as Cana-Vertix® energy cane from GranBio. In other embodiments, the biomass is a starch-containing biomass such as corn, wheat and root crops, or a lignocellulosic biomass, including crop residues (such as cane bagasse and straws of cereals), woods, herbaceous biomass, agro-industrial residues (such as sawdust and wood chips) and cellulosic wastes (such as newsprints and waste office papers).

When the biomass is a carbohydrate-containing biomass, the reaction of the step (A) consists advantageously in a fermentation of the carbohydrate contained in the carbohydrate-containing biomass. Then, the step (A) comprises preferably a separation from the carbohydrate-containing biomass, of a material comprising a higher amount of carbohydrate than the amount of carbohydrate contained in the carbohydrate-containing biomass as a whole, followed by a fermentation of said material to cause the conversion of the carbohydrate contained therein into ethanol.

As above indicated, an exemplary carbohydrate-containing biomass is sugarcane. A sugarcane comprises a stalk comprising a rind and, surrounded by the rind, a pith. The pith (also commonly referred to as sugar-fith, Comfith or derinded stalk) is a matrix of parenchyma cells in which vascular bundles are embedded. A sugarcane comprises generally sugar, especially sucrose, principally in the pith, more precisely in the parenchyma cells. These cells can be easily ruptured to free a juice comprising sugar, advantageously with a high content.

When the biomass is a sugarcane, the step (A) comprises generally the separation from the sugarcane of the pith comprising a higher amount of sugar than the amount of sugar contained in the sugarcane as a whole. Commonly employed methods to separate the pith from the sugarcane are (i) milling and/or crushing, (ii) hot water extraction and/or diffusion, or a combination of (i) and (ii). In the diffusion method, cane is prepared by knife mills and roller crusher combinations. After separation from the sugarcane, the pith comprises typically some parenchyma cells in which vascular bundles are embedded (as before the separation) and a liquid juice comprising sugar, especially sucrose, freed by the rupture of some other parenchyma cells (hereinafter, “pith juice”).

Following its separation from the carbohydrate-containing biomass, the material comprising a higher amount of carbohydrate is advantageously fermented in a reactor, generally a batch reactor, called “fermentor” to cause the conversion of the carbohydrate contained therein into ethanol. For example, following its separation from the sugarcane, the pith is advantageously fermented in a fermentor to cause the conversion of the sugar contained therein into ethanol.

To this end, a fermentation medium, which is typically in the form of a suspension, is advantageously prepared by mixing the material comprising a higher amount of carbohydrate with water. For example, a fermentation medium, which is typically in the form of a suspension, is advantageously prepared by mixing the pith with water.

The mixing can be achieved in a dedicated vessel or directly in the fermentor. The material comprising a higher amount of carbohydrate to water ratio, for example the pith to water ratio, is advantageously such that it allows for the formation of a suspension wherein the fermentation reaction proceeds efficiently. This ratio ranges generally from 0.01 to 2 w/w. Preferably, it ranges from 0.1 to 1 w/w. More preferably it ranges from 0.2 to 0.6 w/w.

The fermentation of the material comprising a higher amount of carbohydrate, for example the pith, is advantageously carried out using a microorganism. So, the fermentation medium advantageously further comprises such a microorganism, in addition to the material comprising a higher amount of carbohydrate (e.g. the pith) and the water. Preferably, the microorganism is a yeast, a bacteria or a fungus. More preferably, the microorganism is a yeast selected from the group consisting of Saccharomyces spp. and Brettanomyces custersii. Still more preferably, it is Saccharomyces cerevisiae. The most preferably, it is Saccharomyces cerevisiae CBS 2959 strain. The fermentation medium is advantageously formed by adding the microorganism to a mixture, typically a suspension, comprising water and the material comprising a higher amount of carbohydrate (e.g. the pith).

When the biomass is a carbohydrate-containing biomass, in particular a sugar, the weight amount of the microorganism, based on the total weight of the carbohydrate, ranges generally from 0.1% to 10%, preferably from 0.3% to 3%.

The fermentation of the material comprising a higher amount of carbohydrate, for example the fermentation of the pith, is advantageously carried out at an acidic pH. So, the fermentation medium comprises advantageously an acid, in addition to the material comprising a higher amount of carbohydrate, the water and the microorganism. The nature and amount of the acid is advantageously chosen in such a way that the fermentation medium has a pH from 2.5 to 5.5, preferably from 3.0 to 5.0, more preferably from 4.0 to 4.5. The nature and amount of the acid is not particularly critical provided they allow for the preparation of a fermentation medium having the desired pH. Non limitative examples of acids suitable for use in accordance with the present invention include hydrochloric acid, nitric acid, sulfuric acid, phosphoric acid, formic acid and acetic acid. The acid is preferably a strong acid, more preferably hydrochloric acid. The fermentation medium is advantageously formed by adding concomitantly the microorganism and the acid to a mixture, typically a suspension, comprising water and the material comprising a higher amount of carbohydrate (e.g. the pith).

Ethanol can be further added to the fermentation medium, generally before the fermentation takes place. The presence of a higher ethanol concentration in the fermentation medium can facilitate the conversion of the biomass. Advantageously, the fermentation takes place at a temperature of from 10° C. to 80° C., preferably of from 15° C. to 60° C. and more preferably of from 20° C. to 40° C.

Generally, the fermentation takes place at a pressure of from 0.1 to 10 MPa, preferably from 0.5 to 5 MPa. More preferably, the reaction takes place at about the atmospheric pressure (1 atm=101.325 kPa). The reaction takes place advantageously in the presence of oxygen, preferably in an air atmosphere.

Usually, the fermentation takes place for a fermentation period of from 10 h to 100 h, preferably of from 20 h to 70 h and more preferably of from 30 h to 60 h.

As above explained, the step (A) comprises generally (A-1) causing a biomass to undergo a chemical transformation in a first reaction medium to produce ethanol, in particular causing a sugarcane pith to undergo a fermentation reaction in a fermentation medium comprising the sugarcane pith and water to produce ethanol; the fermentation medium obtained upon completion (A-1) may comprise the so-produced ethanol, the unfermented pith, the water, the microorganism and the acid.

Advantageously, following (A-1), the step (A) further comprises (A-2) separating the ethanol from the first reaction medium, in particular separating the so-produced ethanol from the fermentation medium comprising the unfermented pith, the water, the microorganism and the acid.

The so-produced ethanol can be separated from the first reaction medium using one or more methods well known to a person skilled in the art. Such methods include but are not limited to sieving, filtration, centrifugation, distillation, adsorption, solvent extraction and pervaporation. A preferred method is or includes filtration. When the biomass is sugarcane and the reaction is a fermentation reaction, the unfermented pith may comprise a fraction of solid particles which can be retained by a filter or a sieve, while the ethanol passes through the filter or the sieve, typically together with the unfermented pith juice, the water, the microorganism and the acid.

When the biomass is a carbohydrate-containing biomass and the first reaction is a fermentation, a particular embodiment of the present invention provides that part or all of the produced ethanol can be recycled, meaning that part or all of the produced ethanol can be added to a new first reaction medium comprising a biomass, and the new first reaction medium can then be caused to undergo a new chemical transformation so as to produce an additional amount of ethanol. It can be proceeded with such a recycling one or several times. As above indicated, the presence of a higher ethanol concentration in the reaction medium can facilitate the conversion of the biomass. When the biomass is sugarcane and the reaction is a fermentation reaction, the ethanol is advantageously recycled with other ingredients comprised in the fermentation medium after the reaction has taken place, such as the unfermented pith juice, the water, the microorganism and the acid.

In the step (A), when the biomass is a carbohydrate-containing biomass, the ethanol is produced in a weight amount advantageously of at least 30 wt %, preferably at least 40 wt %, more preferably at least 50 wt %, still more preferably at least 55 wt % and the most preferably at least about 60 wt %, based on the weight of the carbohydrate contained in the biomass.

According to the step (B), the ethanol produced at the step (A) is reacted with methanol to produce benzyl alcohol and/or benzaldehyde.

This reaction takes advantageously place in the presence of catalyst, which is generally supported on a support. The catalyst is preferably a transition metal catalyst, which is generally supported on a support.

The transition metal for use as the catalyst in the step (B) may be an element chosen from the elements of the d-block of the periodic table, which consists of groups 3 to 12, and the elements of the f-block of the periodic table, which consists of lanthanides and actinides. The transition metal is preferably an element of the d-block of the periodic table; it is more preferably chosen from cobalt, nickel, copper, silver, iridium, zinc and yttrium; still more preferably, it is chosen from cobalt, copper and nickel; the most preferably, it is cobalt.

The transition metal may be provided directly in metal state, with an oxidation state of 0. Alternatively, the transition metal may be embedded in a precursor, typically a metal salt such as a nitrate, a chloride, a levulinate, a sulfate or an acetate salt, wherein the oxidation state of the transition metal is above 0, possibly +1, +2, +3 or +4, preferably +2.

The catalyst, in particular the transition metal catalyst, may be unsupported. Alternatively and preferably, the catalyst, in particular the transition metal catalyst, is supported on a support. As suitable supports of the catalyst, it can be notably cited fumed or colloidal silica, ceramics, metal aluminates, metal silicates, metal aluminosilicates (such as zeolites), metal oxides, metal hydroxides, wherein the metal can be, for example, an alkaline earth metal or a transition metal such as lead. It can also be cited metal (hydrogeno) phosphates, metal hydroxy (hydrogeno) phosphates (such as hydroxyapatites) and metal halogenophosphates (such as fluoroapatites and chloroapatites), wherein the brackets surrounding “hydrogeno” denote that the aforesaid metal compounds can include one or more phosphates and/or one or more hydrogenophosphates groups (HPO₄⁻) and wherein the metal can be, for example, an alkaline earth metal or a transition metal such as lead. In a preferred embodiment, the support is an apatite, especially an apatite chosen from hydroxyapatites, chloroapatites and fluoroapatites; the apatite, especially the hydroxyapatite, the chloroapatite or the fluoroapatite, can be a stoichiometric or non-stoichiometric compound. In another preferred embodiment, the support is a stoichiometric compound of chemical formula MgₓCa_{y}Sr_{z}BaₘPbₙ(OH)ₐCl_{b}F_{c}(PO₄)_{d} wherein x, y, z, m, n, a, b and c are integers greater than or equal to 0, d is an integer greater than 0, x+y+z+m+n is greater than 0, a+b+c is greater than 0 and wherein 2x+2y+2z+2m+2n-a-b-c-3d=0, especially a stoichiometric compound of chemical formula Ca_{y}(OH)ₐCl_{b}F_{c}(PO₄)_{d} wherein y and d are integers greater than 0, a, b and c are integers greater than or equal to 0, a+b+c is greater than 0 and wherein 2y-a-b-c-3d=0. More preferably, the support is a hydroxyapatite of formula Ca₁₀(OH)₂(PO₄)₆. Good results are obtained when the catalyst of the step (B) is cobalt supported on a support which is a hydroxyapatite of formula Ca₁₀(OH)₂(PO₄)₆, commonly referred to as a Co/HAP catalyst.

When the catalyst, in particular the transition metal catalyst, is supported on a support, its loading is generally from 0.1 wt % to 2 wt %, preferably from 0.5 wt % to 1.5 wt %, based on the weight of the support.

Before its use at the step (B), the catalyst is advantageously pre-treated in a hydrogen/inert gas mixed atmosphere, generally at a temperature of from 300°° C. to 700° C., preferably from 350° C. to 450° C. The inert gas is generally nitrogen. The concentration of the hydrogen atmosphere is advantageously of from 3% to 15% H₂/inert gas. Preferably, the concentration of the hydrogen atmosphere is of from 5% to 10% of H₂/inert gas.

The step (B) comprises generally (B-1) causing the ethanol which has been separated from the first reaction medium to react with methanol in a second reaction medium. The reaction of the step (B) can take place in a batch or continuous reactor. Preferably, the reactor is a continuous reactor, more preferably a packed-bed reactor. When the reaction takes place in a continuous reactor, in particular in a packed-bed reactor, the ethanol and the methanol are continuously fed in the reactor.

In general, the reaction takes place in the second reaction medium at a pressure ranging from 0.1 to 10 MPa, preferably from 0.5 to 5 MPa. Preferably, the reaction takes place at about the atmospheric pressure (1 atm=101.325 kPa). The reaction takes place advantageously in an inert atmosphere, preferably in a nitrogen atmosphere. The total partial pressure of ethanol and methanol in the second reaction medium usually ranges from 1 to 20 kPa, preferably from 3 to 12 kPa and more preferably from 4 to 8 kPa.

The initial methanol to ethanol weight ratio, viz. the weight ratio before the methanol and the ethanol are reacted together, usually ranges from 0.5 to 5, often from 0.8 to 3. It is preferably of at least 1, more preferably of at least 1.1 and still more preferably of at least 1.2. Besides, it is preferably of at most 2 and more preferably of at most 1.5. When the second reaction takes place in a reactor wherein the ethanol and the methanol are continuously fed, the molar ratio of ethanol and methanol can be adjusted by controlling the flow rate of the ethanol and methanol.

At the step (B), methyl benzaldehyde and/or methyl benzyl alcohol can be formed as by-products; by co-feeding ethanol and methanol with an appropriate methanol to ethanol weight ratio as above detailed, a higher selectivity towards the benzyl alcohol and/or benzaldehyde can be achieved. In contrast, if the ethanol were fed alone, methyl benzaldehyde and/or methyl benzyl alcohol would be formed as the only aromatic products.

In general, the reaction of the step (B) takes place at a temperature of from 200° C. to 500° C. Preferably, the temperature of the reaction ranges from 300° C. to 400° C. More preferably, it ranges from 325° C. to 375° C.

The catalytic conversion takes place for a time period (in case of a batch reaction) or residence time (in case of a continuous process) which usually ranges from 0.01 h to 100 h, and often from 0.1 h to 10 h.

The weight amount of ethanol which is converted during the step (B) is generally of at least 10 wt %, preferably at least 15%, and may be of at least 30%, at least 45% or at least 60%, at least 75% or at least 90%. Indeed, any unconverted ethanol, after separation of the reaction products as will be detailed later on, can be re-used as reactant of a new second reaction medium, and the unconverted ethanol can be recycled as many times as desired, possibly until it is essentially fully converted.

In general, both benzyl alcohol and benzaldehyde are produced in the step (B). The weight amount of the benzyl alcohol, based on the combined weight amount of the benzyl alcohol and the ethanol, may vary to some extent depending notably on the temperature at which the ethanol and the methanol are reacted; it ranges generally from 50 wt % up to less than 100 wt %, very often from 70 wt % to 95 wt %, possibly from 85 wt % to 95 wt %.

Advantageously, following the sub-step (B-1), the step (B) further comprises (B-2) separating the benzyl alcohol and/or benzaldehyde from the second reaction medium; more specifically, the sub-step (B-2) allows typically for the separation of benzyl alcohol and/or benzaldehyde from the unconverted ethanol, the unconverted methanol and unwanted reaction by-products, in particular 2-methyl benzyl alcohol, 4-methylbenzyl alcohol, 2-tolualdehyde and 4-tolualdehyde. The sub-step (B-2) can be achieved by using one or more methods well known to a person skilled in the art. A preferred method is or includes distillation. The distillation is advantageously operated using a single distillation column. The distillation column may operate at a temperature of from 100° C. to 200° C., preferably at a temperature of from 110° C. to 170° C., more preferably at a temperature of from 120° C. to 160° C. The distillation column may operate under vacuum.

Preferably, the sub-step (B-2) provides further that the unconverted ethanol is separated from the second reaction medium and that the so-separated unconverted ethanol is recycled, that is to say that it is re-used as reactant of a new second reaction medium in accordance with the sub-step (B-1); the unconverted ethanol can be recycled as many times as desired, possibly until it is fully or essentially fully converted. Also preferably, the sub-step (B-2) provides further that the unconverted methanol is separated from the second reaction medium and that the so-separated unconverted methanol is recycled, that is to say that it is re-used as reactant of a new second reaction medium in accordance with the sub-step (B-1); the unconverted methanol can be recycled as many times as desired, which can be one or several times, possibly until it is fully or essentially fully converted. Very preferably, the sub-step (B-2) provides further that the unconverted ethanol and the unconverted methanol are separated from the second reaction medium and that the so-separated unconverted ethanol and methanol are recycled, that is to say that they are re-used as reactants of a new second reaction medium; the unconverted ethanol and the unconverted methanol can be recycled as many times as desired, possibly until they are fully or essentially fully converted.

In the step (B), in the absence of any unconverted ethanol recycling, the benzyl alcohol and/or the benzaldehyde are produced in a combined weight amount, viz. the weight of the benzyl alcohol plus the weight of the benzaldehyde, of advantageously of at least 10 wt %, possibly at least 15 wt % or at least 20 wt %, based on the weight of the ethanol which is used as reagent.

In the step (B), the selectivity in the benzyl alcohol and/or the benzaldehyde can exceed 20%, 25%, 30% or even 35%. So, when the unconverted ethanol is recycled and further reacted, desirably until it is fully or essentially fully converted, the benzyl alcohol and/or the benzaldehyde can be produced in a combined weight amount of at least 20 wt %, possibly at least 25 wt %, at least 30 wt % or at least 35 wt %, based on the initial weight of the ethanol.

According to the step (C), the benzyl alcohol and/or the benzaldehyde produced at the step (B) are caused to react with oxygen, so as to produce benzoic acid.

The reaction of the step (C) is typically an oxidation reaction.

The oxygen which is reacted during the step (C) may be provided as such (that is to say unmixed with other gases such as nitrogen and rare gases) or in admixture with one or more inert gases. The reaction of the step (C) is advantageously performed in an air atmosphere, wherein the air comprises oxygen to be caused to react with the benzyl alcohol and/or the benzaldehyde. The case being, the reaction of the step (C) is performed at an air pressure which is advantageously in the range of 0.1 to 10 bar, preferably from 0.5 to 2 bar. More preferably, the reaction of the step (C) is conducted at about the atmospheric pressure (1 atm=101.325 kPa).

The reaction of the step (C) is advantageously performed in the presence of a catalyst, which is generally supported on a support. Preferably, the reaction of the step (C) is advantageously performed in the presence of a catalyst and of a co-catalyst, which are generally supported on a support.

The catalyst is preferably a transition metal catalyst, which is generally supported on a support.

The transition metal for use as catalyst in the step (C) may be an element chosen from the elements of the d-block of the periodic table, which consists of groups 3 to 12, and the elements of the f-block of the periodic table, which consists of lanthanides and actinides. Its electronegativity is advantageously of at least 1.9, as it is the case for noble metals and also for silver (Ag), rhenium (Re), copper (Cu) and mercury (Hg). Preferably, the transition metal is a noble metal. As herein used, the term “noble metal” denotes any element chosen from ruthenium (Ru), rhodium (Rh), palladium (Pd), osmium (Os), iridium (Ir), platinum (Pt) and gold (Ag). More preferably, the transition metal is chosen from ruthenium, rhodium, palladium, iridium and platinum. Still more preferably, the transition metal is platinum.

The co-catalyst is preferably a poor metal catalyst, which is generally supported on a support. Also preferably, the co-catalyst is supported on the same support as the catalyst.

The poor metal for use as catalyst in the step (C) is to be found among the elements of group 12 to 16 of the periodic table of the elements, with the proviso that it differs from an element of group 12 when the transition metal for use as catalyst in the step (C) is an element of such group 12. As herein used, the term “poor metal” denotes any element chosen from zinc (Zn), cadmium (Cd), mercury (Hg), aluminum (Al), gallium (Ga), indium (In), thallium (TI), germanium (Ge), tin (Sn), lead (Pb), antimony (Sb), bismuth (Bi), tellurium (Te) and polonium (Po). Among them, aluminum, gallium, indium, thallium, tin, lead, bismuth and polonium are commonly referred to as “post-transition metals”. The poor metal is preferably chosen from aluminum, gallium, indium, thallium, germanium, tin, lead, antimony, bismuth and polonium. More preferably, the poor metal is chosen from tin, lead, antimony, bismuth and polonium. Still more preferably, the poor metal is bismuth.

The catalyst (in particular, the transition metal catalyst) and, when present, the co-catalyst (in particular, the poor metal co-catalyst) may be unsupported. Alternatively and preferably, the catalyst (in particular, the transition metal catalyst) and, when present, the co-catalyst (in particular, the poor metal co-catalyst) are supported on a support. As suitable supports of the catalyst and, when present, the co-catalyst, it can be notably cited zeolites, Kieselguhr, silica, alumina, silica-alumina, clay, titania, zirconia, magnesia, calcia, lanthanum oxide, niobium oxide and activated carbon. Preferably, the support is chosen from the group consisting of activated carbon, alumina, clay and titania. More preferably, the support of the catalyst and, when present, the co-catalyst is activated carbon.

Good results are obtained when the catalytic system of the step (C) is a platinum catalyst combined with a bismuth co-catalyst, both being supported on a support which is activated carbon. This catalytic system is commonly referred to as a Pt—Bi/C catalyst.

The weight amount of the catalyst (in particular, the transition metal catalyst) which is advantageously used in the reaction of the step (C) typically from 1 wt % to 10 wt %, preferably from 3 wt % to 7 wt %, more preferably from 4 wt % to 6 wt %, based on the combined weight of the benzyl alcohol and the benzaldehyde.

The weight amount of the co-catalyst (in particular, the poor metal catalyst) which is preferably used in combination with the catalyst in the reaction of the step (C) ranges typically from 10 wt % to 100 wt %, preferably from 15 wt % to 70 wt %, more preferably from 20 wt % to 50 wt %, based on the weight of the catalyst.

When the catalyst, in particular the transition metal catalyst, is supported on a support, its loading is generally from 0.05 wt % to 10 wt %, preferably from 0.1 wt % to 2 wt %, based on the weight of the support.

The reaction of the step (C) is advantageously performed in the presence of a base, such as K₂CO₃, NH₃ or a strong base. The base is preferably a strong base, such as Ca(OH)₂, Al(OH)₃ or an alkali metal hydroxide. The base is more preferably an alkali metal hydroxide, such as KOH, NaOH or LiOH. Still more preferably, the base is KOH.

The weight amount of the base used in the reaction ranges generally from 10 wt % to 1000 wt %, preferably from 100 wt % to 500 wt %, based on the combined weight of the benzyl alcohol and/or the benzaldehyde.

The reaction of the step (C) is advantageously performed in the presence of a solvent. The solvent used in the reaction of the step (C) is typically water, a C₁-C₁₂ aliphatic alcohol or a mixture thereof. It is preferably water, a C₁-C₅ alkanol or a mixture thereof. As the C₁-C₅ alkanol, methanol and ethanol are preferred. More preferably, the solvent is a mixture of methanol and water.

Advantageously, the reaction of the step (C) takes place at a temperature of from 10° C. to 100° C., preferably of from 30° C. to 80° C., more preferably of from 40° C. to 60° C.

Usually, the reaction of the step (C) takes place for a time period of from 30 min to 30 h, preferably from 1 to 10 h. The time period during which the reaction of the step (C) takes place allows advantageously for reaching a conversion into benzoic acid of at least 80 wt %, preferably at least 90 wt %, more preferably at least 95 wt %, based on the combined weight of the benzyl alcohol and/or the benzaldehyde.

As above detailed, the step (C) comprises generally (C-1) causing the benzyl alcohol and/or the benzaldehyde which have been separated from the second reaction medium to react with oxygen in a third reaction medium, so as to produce benzoic acid in the third reaction medium.

The resulting third reaction medium is advantageously acidified using an acid. Non limitative examples of acids suitable for acidifying the resulting reaction medium include hydrochloric acid, nitric acid, sulfuric acid, phosphoric acid, formic acid and acetic acid. The acid is preferably a strong acid, more preferably hydrochloric acid.

After the acidification, the step (C) comprises desirably (C-2) separating the benzoic acid from the third reaction medium. The so-produced benzoic acid can be separated from the third reaction medium using one or more methods well known to a person skilled in the art. Such methods include but are not limited to filtration, centrifugation, distillation, adsorption, solvent extraction and pervaporation. Usually, the catalyst is firstly separated from the resulting reaction mixture, preferably by centrifugation. The remaining liquid part is then advantageously extracted with a solvent, preferably an ester, more preferably an alkyl alkanoate with ethyl acetate being especially recommended. Thereafter, the benzoic acid is advantageously separated from the so-extracted liquid, using preferably a distillation column under vacuum. The temperature in the distillation column may range from 125° C. to 250° C., possibly from 150° C. to 225° C. or from 170° C. to 200° C. Alternatively, the benzoic acid may be separated directly from the remaining liquid part (without further solvent extraction) or directly from the raw third reaction medium resulting from (C-1).

In the step (C), the benzoic acid is produced in a weight amount of advantageously at least 80 wt %, preferably at least 90 wt % and more preferably at least 95 wt %, based on the combined weight of the benzyl alcohol and the benzaldehyde.

Summarizing all the above, the steps (A), (B) and (C) of the process in accordance with the present invention can be characterized in that:
the step (A) comprises (A-1) causing a biomass to undergo a chemical transformation in a first reaction medium, so as to produce ethanol in the first reaction medium, then (A-2) separating the ethanol from the first reaction medium;
the step (B) comprises (B-1) causing the so-separated ethanol to react with methanol in a second reaction medium, so as to produce benzyl alcohol and/or benzaldehyde in the second reaction medium, then (B-2) separating the benzyl alcohol and/or benzaldehyde from the second reaction medium; and
the step (C) comprises (C-1) causing the so-separated benzyl alcohol and/or benzaldehyde to react with oxygen in a third reaction medium, so as to produce benzoic acid in the third reaction medium, then (C-2) separating the benzoic acid from the third reaction medium.

The benzoic acid synthesized by the process according to the present invention can be used as such as an expectorant, as an analgesic, as an antiseptic or as an antimicrobial agent.

The benzoic acid synthesized by the process according to the present invention can also be used for the preparation of a benzoic acid derivative.

As a first example, it can be used for the synthesis of ε-caprolactam. A method for producing ε-caprolactam from benzoic acid is described in CN102060767A (assigned to Dalian Institute of Chemical Physics of CAS), the whole content of which is herein incorporated by reference.

As another example, it can be used for the synthesis of a salt or ester of benzoic acid. Accordingly, the present invention also concerns a method for the synthesis of a salt or ester of benzoic acid, said method comprising:
producing benzoic acid by the process as above described, then
causing the so-produced benzoic acid to be either neutralized by a Brönsted base or esterified by an alcohol.

In a first embodiment of the invented method, the benzoic acid is neutralized by a Brönsted base so as to synthesize a salt of benzoic acid.

The Brönsted base is preferably a monohydroxide of formula X—OH or a dihydroxide of formula HO—Y—OH, wherein X represents an alkali metal (such as Na, K or Li) or an ammonium group and Y represents a divalent metal, in particular an alkaline earth metal (such as Ca). Other Brönsted bases can be used such as Na₂CO₃ or K₂CO₃.

More preferably, the salt of benzoic acid is sodium benzoate.

Still more preferably, the Brönsted base is NaOH and the salt of benzoic acid is sodium benzoate.

In another embodiment of the invented method, the benzoic acid is esterified by an alcohol so as to synthesize an ester of benzoic acid.

The alcohol is preferably a monoalcohol of formula R-OH or a diol of formula HO—Z—OH, wherein R represents a C₁-C₂₀ alkyl group and Z represents a C₂-C₂₀ alkanediyl group or a group of formula

-A-(—O-A-)ⱼ—O-A-
wherein A is a C₁-C₅ alkanediyl group (such as —CH₂—CH₂— or —CH(CH₃)—CH₂—) and j is an integer ranging from 0 to 4 (such as 0, 1 or 2).

As examples of suitable alcohols, it can be cited methanol, ethanol, n-propanol, isopropanol, n-butanol, isobutanol, n-pentanol, isoamyl alcohol, n-hexanol, n-octanol, 2-ethyl-1-hexanol, isononyl alcohol, isodecyl alcohol, lauryl alcohol, myristyl alcohol, cetyl alcohol, 2-hexyl-1-decanol, stearyl alcohol, ethylene glycol, diethylene glycol, triethylene glycol, propylene glycol, dipropylene glycol, tripropylene glycol and 2,2-dimethylpropane-1,3-diol.

A preferred alcohol for the esterification of benzoic acid is methanol and the product of the esterification of benzoic acid is then methyl benzoate.

In a particular embodiment, the methyl benzoate is further transesterified with a monoalcohol of formula R′—OH or a diol of formula HO—Z—OH, wherein R′ represents a C₂-C₂₀ alkyl group and Z represents a C₂-C₂₀ alkanediyl group or a group of formula

-A-(—O-A-)ⱼ—O-A-
wherein A is a C₁-C₅ alkanediyl group (such as —CH₂—CH₂— or —CH(CH₃)—CH₂—) and j is an integer ranging from 0 to 4 (such as 0, 1 or 2), so as to obtain an ester of benzoic acid other than methyl benzoate.

As the result of the esterification or transesterification of benzoic acid, various esters can be synthesized, among which 2-ethylhexyl benzoate, isononyl benzoate, isodecyl benzoate, 2-hydroxyethyl benzoate, 2-(2-hydroxyethoxy) ethyl benzoate (also named “diethylene glycol benzoate”), 2-(benzoyloxy) ethyl benzoate (also named “ethylene glycol dibenzoate”), diethylene glycol dibenzoate, triethyleneglycol dibenzoate, 2-(benzoyloxy) propyl benzoate (also named “propylene glycol dibenzoate”), dipropylene glycol dibenzoate, tripropyleneglycol dibenzoate and 2,2-dimethylpropane-1,3-diyl dibenzoate (also named “neopentyl glycol dibenzoate”) are worth being cited.

The process in accordance with the present invention has numerous advantages. It provides a sustainable way of producing benzoic acid from a renewable biomass: it is fully capable of producing benzoic acid from real biomass, in its whole complexity. It uses advantageously low amounts of catalysts. The benzoic acid produced by the process of the present invention can be easily separated from its reaction medium, and the same holds true for the intermediates produced at the different steps of the invented process, viz. the ethanol and the benzyl alcohol and/or the benzaldehyde. The benzoic acid can be obtained with a reasonably high yield, advantageously exceeding 20 wt % (as herein used, the yield is the ratio of the weight of the produced benzoic acid to the weight of the useful reagent fraction contained in the biomass, typically the carbohydrate fraction). All in all, it is contemplated that the presently invented process, is able to combine outstandingly the “green” attribute, viz. the use of a renewable biomass as starting reagent, with an excellent viability at industrial scale and a reasonably high economic attractiveness.

Should the disclosure of any patents, patent applications, and publications which are incorporated herein by reference conflict with the description of the present application to the extent that it may render a term unclear, the present description shall take precedence.

### EXAMPLES (In Accordance With The Invention)

### Materials

Ca(NO₃)₂·4H₂O, CAS No. 13477-34-4, Sinopharm; (NH₄)₂HPO₄, CAS No. 7783-28-0, Sinopharm; NH₃·H₂O, CAS No. 7664-41-7, 25 wt % in H₂O, Sinopharm; Co(NO₃)₂·6H₂O, CAS No. 10026-22-9, Sinopharm; Ni(NO₃)₂·6H₂O, CAS No. 13478-00-7, Sinopharm; Fe(NO₃)₃·6H₂O, CAS No. 7782-61-8, Sinopharm; NaOH, CAS No. 1310-73-2, Sinopharm; Na₂CO₃, CAS No. 497-19-8, Sinopharm; NH₄VO₃, CAS No. 7803-55-6, Sinopharm; Bio-ethanol, CAS No. 64-17-5, CREMER OLEO GmbH & Co. KG; Bio-methanol, CAS No. 67-56-1, Merck; Benzaldehyde (BA=O), CAS No. 100-52-7, J&K; Benzyl alcohol (BA-OH), CAS No. 100-51-6, J&K; 2-Methylbenzyl alcohol (MB—OH), CAS No. 89-95-2, J&K; 4-Methylbenzyl alcohol (MB—OH), CAS No. 589-18-4, J&K; Propanol, CAS No. 71-23-8, J&K; Butanol, CAS No. 71-36-3, J&K; Pentanol, CAS No. 71-41-0, J&K; Hexanol, CAS No. 111-27-3, J&K; Acetaldehyde, CAS No. 75-07-0, J&K; E-2-butenal, CAS No. 123-73-9, J&K; 2-Tolualdehyde (2-MB═O), CAS No. 529-20-4, J&K; 4-Tolualdehyde (4-MB═O), CAS No. 104-87-0, J&K; 5 wt % Pt-1.5 wt % Bi/C (moisture: 46.8%), Type 160, CAS No. 7440-06-4, Johnson Matthey; KOH, CAS No. 1310-58-3, 95% purity, Sinopharm; HCl, CAS No. 7647-01-0, 37 wt %, Sinopharm; Ethyl acetate, CAS No. 141-78-6, Sinopharm.

### Synthesis of HAP

Hydroxyapatite (HAP) is synthesized by a precipitation method. An aqueous solution of Ca(NO₃)₂·4H₂O (0.6 M, Sinopharm) is added dropwise to a solution of (NH₄)₂HPO₄ (0.4 M, Sinopharm). Then, NH₃·H₂O (25 wt % in H₂O, Sinopharm) is added to the solution to achieve an initial system having a pH of 10.3. The slurry is stirred for 24 h at 80° C. After filtration, drying, and calcination (600° C. for 2 h in static air), a white HAP is obtained. The overall Ca/P ratio is about 1.67, as measured using an inductively-coupled plasma device (ICP).

### Synthesis of Co/HAP Catalyst

HAP is impregnated with an 0.35 M aqueous solution of Co(NO₃)₂·6H₂O by incipient wetness impregnation. The slurry is stirred at room temperature for 30 min. After drying at 50° C. for 12 h in air and calcination at 350° C. for 2 h in air, the as-prepared Co/HAP catalyst is obtained. The actual Co content is about 0.8 wt %, as determined by ICP, and the chemical state of Co is +2, as determined by XPS.

### Synthesis of NiO—Fe₂O₃—Na₂O—V₂O₅ Catalyst

NiO—Fe₂O₃ complex oxide catalyst is prepared by co-precipitation using an aqueous solution of Ni(NO₃)₂·6H₂O, Fe(NO₃)₃·6H₂O and NaOH. 144 g of Ni(NO₃)₂·6H₂O and 101 g of Fe(NO₃)₃·6H₂O are dissolved in 400 mL of pure water, and about 100 g of NaOH are dissolved in 500 mL of pure water. Both solutions are added dropwise to 2 L of pure water at room temperature so as to maintain the system at a pH in the range of 7-8. After adding the solutions, the mixture is stirred at room temperature for about 1 h. The precipitates obtained are washed with pure water until they become free from sodium anion, then dried at 110°° C. for 24 h in air. After calcination at 800° C. for 3 h in air, the catalysts are pulverized to particles in the range of 20-40 mesh.

Modifications with Na₂O and V₂O₅ are performed using an impregnation method with an aqueous solution of Na₂CO₃ and NH₄VO₃. 100 mL of an aqueous solution containing 1.37 g of Na₂CO₃ and 1.55 g of NH₄VO₃ are added to the above NiO—Fe₂O₃ complex oxide catalyst, and stirred at room temperature for about 1 h. The precipitates obtained are filtered and dried in the atmosphere at 120° C. for 24 h, then calcined in the atmosphere at 800° C. for 4 h to obtain a catalyst having a weight ratio of about 46:50:1:3 as NiO—Fe₂O₃—Na₂O—V₂O₅.

### Fermentation of Sugar Cane to Ethanol, in Accordance with Step (A) of the Invented Process

Freshly cut cane stalks are milled in a cane separator and the pith is recovered, representing about 80% by weight of the fresh cane. Approximately 150 kg of the pith are placed in a 1000 L vessel together with 400 L of tap water. The pH is adjusted to a value of 4.0-4.5 with a 10 N solution of HCl and the whole mixture is cooled and inoculated with a 24-h old inoculum of a strain of Saccharomyces cerevisae (yeast), viz. CBS 2959 strain. The volume of inoculum is 80 L, with about 15 g/L of total sugars, about 28 g/L of ethanol and about 9 g/L of dry biomass. The vessel is incubated at 30° C. for 40 h without agitation. An ethanolic-yeast suspension is obtained. Solid cane pieces or particles are thereafter separated from the ethanolic-yeast suspension by a filter. The resulting filtrate has an ethanol concentration of about 27 g/L, a total sugars concentration of about 0.2 g/L and a dry biomass concentration of about 3 g/L. About 14.4 kg of ethanol are produced. The consumption of extractable sugars is above 99%; the ethanol yield is about 0.60 kg ethanol/kg sucrose consumed; the total yeast biomass produced is about 1.2 kg and the yeast yield is about 0.05 kg dry biomass/kg sucrose consumed. About 340 L of this ethanolic-yeast suspension are then mixed with about 30 kg of cane pith previously dried in a forced-air tray dryer employing air at 60° C. to reach a final moisture of about 2%. The vessel is incubated at 30° C. for 24 h. After that time, an ethanol-yeast suspension is again separated by filtration. The composition of the resulting filtrate is about 50 g/L of ethanol, about 0.6 g/L of total sugars and about 6 g/L of dry biomass. The ethanol produced in this second fermentation is about 9.4 kg; more than 98% of the total extractable sugars are consumed; the yield is about 0.63 kg ethanol/kg sucrose consumed; the total yeast biomass produced is about 850 g and the yeast yield is about 0.06 kg dry biomass/kg sucrose consumed. In all, the yield is about 60 kg bioethanol/100 kg sucrose.

### Conversion of Benzyl Alcohol and Benzaldehyde into Ethanol, in Accordance with Step (B) of the Invented Process

### Catalytic Reaction at 325° C.

Catalytic reaction is carried out in a packed-bed reactor. The reaction temperature is maintained using a vertically aligned tube furnace equipped with a thermocouple.

150 g of 0.8 wt % Co/HAP catalyst are pre-treated at 400° C. for 2 h in 8% H₂/N₂ prior to the reaction. The reaction is carried out under atmospheric pressure with a total gas flow rate of 30 L/min. For the whole reaction, the feed gas is 4 vol % methanol and 2 vol % ethanol, and is balanced with nitrogen. The weight hourly space velocities of ethanol and methanol are 0.37 g ethanol/_{(g catalyst·h)} and 0.51 g methanol/_{(g catalyst·h)}, respectively. The reaction temperature is set at 325° C. The reaction is operated for several tens of hours at 325° C., during which the Co/HAP exhibits a good stability.

To determine the composition of the organic products exiting the packed bed reactor, these ones are collected in cold traps with acetone and analyzed using an on-line gas chromatograph (GC) equipped with a flame ionization detector. The residence time for each specific component is determined using the corresponding standard chemicals. The identities of the organic products are further confirmed by GC/MS analysis. The organic products include benzaldehyde, benzyl alcohol, unconverted methanol, unconverted ethanol and various by-products, viz. propanol, butanol, pentanol, hexanol, 2-methylbenzyl alcohol, 4-methylbenzyl alcohol, acrolein, acetaldehyde, E-2-butenal, 2-tolualdehyde and 4-tolualdehyde.

Under the above operating conditions, the ethanol conversion achieved after one pass in the reactor ranges from about 38% to about 47%. The selectivity towards the benzyl alcohol is about 22% and the selectivity towards the benzaldehyde is about 4%.

The benzyl alcohol and the benzaldehyde are separated from the unconverted ethanol, the unconverted methanol and the reaction by-products (butanol, pentanol, hexanol, 2-methylbenzyl alcohol, 4-methylbenzyl alcohol, acrolein, acetaldehyde, E-2-butenal, 2-tolualdehyde and 4-tolualdehyde) by distillation. To get rid of the 2-methylbenzyl alcohol, the 4-methylbenzyl alcohol, the 2-tolualdehyde and the 4-tolualdehyde, a distillation under vacuum is operated with a pot temperature of 120-160° C.

The unconverted ethanol and the unconverted methanol are recycled, that is to say they are re-engaged in a new catalytic reaction, in admixture with fresh methanol and ethanol originating from the step (A); the amounts of fresh methanol and ethanol are such that they compensate the respective amounts of methanol and ethanol that have been converted during the first catalytic reaction. The new catalytic reaction is carried out using the same equipment and the same operating conditions as above detailed. The newly obtained benzyl alcohol and benzyl alcohol are again separated from the unconverted ethanol, the unconverted methanol and the reaction by-products by distillation. After this second pass, the recycling of the still unconverted ethanol in the reactor followed by the separation of the reaction products may then be repeated as much as needed to reach finally an ethanol conversion which approaches 100%. By doing so, the combined yield in benzyl alcohol and in benzaldehyde reaches about 250 g/kg ethanol.

All in all, about 2.8 kg of ethanol produced during the step (A) are converted, and about 700 g of benzyl alcohol and benzaldehyde are recovered.

### Catalytic Reaction at 350° C.

The catalytic reaction is carried out using the same equipment and the same operating conditions as above detailed, except that the reaction temperature is set to 350° C. The selectivity towards the benzyl alcohol is about 24% and the selectivity towards the benzaldehyde is about 8%. After recycling so as to reach a final ethanol conversion approaching 100%, the combined yield in benzyl alcohol and in benzaldehyde slightly exceeds 300 g/kg ethanol.

### Catalytic Reaction at 375° C.

The catalytic reaction is carried out using the same equipment and the same operating conditions as above detailed, except that the reaction temperature is set to 375° C. The selectivity towards the benzyl alcohol is about 21% and the selectivity towards the benzaldehyde is about 14%. After recycling so as to reach a final ethanol conversion approaching 100%, the combined yield in benzyl alcohol and in benzaldehyde is about 350 g/kg ethanol.

### Oxidation of Benzyl Alcohol and Benzaldehyde to Benzoic Acid, in Accordance with Step (C) of the Invented Process

4.2 g (0.5 wt % catalyst loading) of 5 wt % Pt-1.5 wt % Bi/C (“160 paste”, moisture 46.8%), 500 g of a mixture of benzaldehyde and benzyl alcohol from the step (B), 1 kg of KOH, 500 mL of methanol, and 4.5 L of pure H₂O are added sequentially into a 10 L reactor. The reactor is then closed and charged with 1 bar of oxygen. Next, the reactor is heated at 50° C. The reaction pressure of O₂ is kept at 1 bar during the whole process. Reaction samples are taken from the reactor and analyzed by GC so as to check if essentially the whole amount of the benzaldehyde and the benzyl alcohol is fully converted. When it is so, the reaction mixture is acidified with 1 N HCl aqueous solution, which is all filtered and transferred in a storage vessel. The yield for benzoic acid is determined by ¹H NMR analysis using 1,3,5-trimethoxybenzene as the internal standard and CD₃OD as deuterated solvent. For the isolation of benzoic acid, after the acidification, the catalyst is separated by centrifugation and the liquid part is extracted by ethyl acetate to obtain the organic solution of benzoic acid. The latter is then distilled under vacuum to obtain 490 g of benzoic acid with a purity of higher than 99% at a temperature of 170-200° C. In all, the yield is 98 g benzoic acid/100 g mixture of benzaldehyde and benzyl alcohol.

1. A process for the production of benzoic acid, said process comprising the steps of:
(A) producing ethanol from a biomass, then
(B) causing the ethanol produced at the step (A) to react with methanol, so as to produce benzyl alcohol and/or benzaldehyde, then
(C) causing the benzyl alcohol and/or benzaldehyde produced at the step (B) to react with oxygen, so as to produce benzoic acid.

2. The process according to claim 1, wherein the biomass contains a carbohydrate, wherein the carbohydrate is a monosaccharide.

3. (canceled)

4. The process according to claim 1, wherein the reaction of the step (A) is a fermentation reaction and the fermentation takes place in a fermentation medium comprising a microorganism, wherein the microorganism is a yeast selected from the group consisting of Saccharomyces spp. and Brettanomyces custersii.

5. (canceled)

6. (canceled)

7. The process according to claim 1, wherein the step (B) takes place in the presence of a transition metal catalyst supported on a support, wherein the transition metal is chosen from cobalt, nickel, copper, silver, iridium, zinc and yttrium, and wherein the support is the apatite hydroxyapatite of formula Ca₁₀(OH)₂(PO₄)₆.

8. (canceled)

9. (canceled)

10. (canceled)

11. (canceled)

12. (canceled)

13. The process according to claim 1, wherein the step (C) takes place in the presence of a transition metal catalyst and, optionally in addition, of a co-catalyst, wherein, the catalyst and, when present, the co-catalyst are supported on a same support.

14. The process according to claim 13, where the transition metal is a noble metal, wherein the noble metal is platinum.

15. (canceled)

16. The process according to claim 13, wherein the co-catalyst is present and is a poor metal, wherein the poor metal is bismuth.

17. (canceled)

18. (canceled)

19. The process according to claim 1, wherein:
the step (A) comprises (A-1) causing a biomass to undergo a chemical transformation in a first reaction medium, so as to produce ethanol in the first reaction medium, then (A-2) separating the ethanol from the first reaction medium;
the step (B) comprises (B-1) causing the so-separated ethanol to react with methanol in a second reaction medium, so as to produce benzyl alcohol and/or benzaldehyde in the second reaction medium, then (B-2) separating the benzyl alcohol and/or benzaldehyde from the second reaction medium; and
the step (C) comprises (C-1) causing the so-separated benzyl alcohol and/or benzaldehyde to react with oxygen in a third reaction medium, so as to produce benzoic acid in the third reaction medium, then (C-2) separating the benzoic acid from the third reaction medium.

20. The process according to claim 19, wherein the sub-step (B-2) further comprises separating the unconverted ethanol from the second reaction medium and recycling it one or several times, that is to say re-using it as reactant of a new second reaction medium in accordance with the sub-step (B-1).

21. The process according to claim 19, wherein the sub-step (B-2) further comprises separating the unconverted methanol from the second reaction medium and recycling it one or several times, that is to say re-using it as reactant of a new second reaction medium in accordance with the sub-step (B-1).

22. A method for the synthesis of a salt or ester of benzoic acid, said method comprising:
producing benzoic acid by the process according to claim 1, then causing the so-produced benzoic acid to be either neutralized by a Brönsted base or esterified by an alcohol.

23. The method according to claim 22, wherein the benzoic acid is neutralized by a Brönsted base so as to synthesize a salt of benzoic acid, wherein the Brönsted base is a monohydroxide of formula X—OH or a dihydroxide of formula HO—Y—OH, wherein X represents an alkali metal or an ammonium group and Y represents a divalent metal, in particular an alkaline earth metal, and wherein the salt of benzoic acid is sodium benzoate.

24. (canceled)

25. (canceled)

26. The method according to claim 22, wherein the benzoic acid is esterified by an alcohol so as to synthesize an ester of benzoic acid, wherein the alcohol is a monoalcohol of formula R—OH or a diol of formula HO—Z—OH, wherein R represents a C1-C20 alkyl group and Z represents a C2-C20 alkanediyl group or a group of formula -A-(—O-A-)j-O-A- wherein A is a C1-C5 alkanediyl group and j ranges from 0 to 4.

27. (canceled)

28. The method according to claim 27, wherein the alcohol is methanol and the ester of benzoic acid is methyl benzoate.

29. The method according to claim 28, wherein the methyl benzoate is further transesterified with a monoalcohol of formula R′—OH or a diol of formula HO—Z—OH, wherein R′ represents a C2-C20 alkyl group and Z represents a C2-C20 alkanediyl group or a group of formula -A-(—O-A-)j-O-A- wherein A is a C1-C5 alkanediyl group and j ranges from 0 to 4, so as to obtain an ester of benzoic acid other than methyl benzoate, wherein the ester of benzoic acid is selected from the group consisting of 2-ethylhexyl benzoate, isononyl benzoate, isodecyl benzoate, 2-hydroxyethyl benzoate, diethylene glycol benzoate, ethylene glycol dibenzoate, diethylene glycol dibenzoate, triethyleneglycol dibenzoate, propylene glycol dibenzoate, dipropylene glycol dibenzoate, tripropyleneglycol dibenzoate and neopentyl glycol dibenzoate.

30. (canceled)
